(19) United States
(12) Patent Application Publication
(10) Pub. No.: US 2022/0071947 A1
(43) Pub. Date: Mar. 10, 2022

(54) PHARMACEUTICAL APPLICATION OF DIMYRICETIN-BASED DISELENIDE

(71) Applicant: Shanghai Spark Pharmaceutical Co., Ltd., Shanghai (CN)

(72) Inventors: KUNYUAN SONG, Shanghai (CN); WEIWEI CHEN, Shanghai (CN)

(21) Appl. No.: 17/297,913

(22) PCT Filed: Jun. 18, 2020
(86) PCT No.: PCT/CN2020/096837

§ 371 Date: May 27, 2021

(30) Foreign Application Priority Data
Apr. 30, 2020 (CN) 202010362352.7

(51) Int. Cl. A61K 31/352; A61P 31/14; A61P 35/00

(52) U.S. Cl. CPC A61K 31/352; A61P 35/00; A61P 31/14

(57) ABSTRACT

The present disclosure provides a pharmaceutical application of dimyricetin-based diselenide. Dimyricetin-based diselenide has the effect of treating tumors and resistance of new coronavirus, and has obvious inhibitory effects on 7 different human-derived tumor cells. The IC50 value of Mpro, the target of the new coronavirus (2019-nCoV), is 0.807±0.0830 μM, which has a significant effect.

### TECHNICAL FIELD

The present disclosure belongs to the technical field of medicine, and specifically relates to a pharmaceutical application of dimyricetin-based diselenide.

### BACKGROUND OF RELATED ARTS

Selenium is a trace element necessary for the human body. It has various biological activities such as anti-oxidation, anti-cancer, anti-cancer, protection of bone marrow hematopoiesis, and delaying aging. At the same time, it has detoxification effect on some heavy metal elements (such as mercury, arsenic, silver, etc.).

Diselenide has an important antioxidant effect, and has the activity of simulating glutathione peroxidase (GSH-PX). Some diselenide also has anti-tumor, antibacterial, bactericidal, and disinfectant activities.

### SUMMARY

The present disclosure is based on the research of diselenide, and aims to provide a pharmaceutical application of dimyricetin-based diselenide with effect of treating tumors and resistance of new coronavirus.

The present disclosure provides an application of dimyricetin-based diselenide represented by molecular structural formula (1) in the treatment of tumors and resistance of new coronavirus (2019-nCoV)

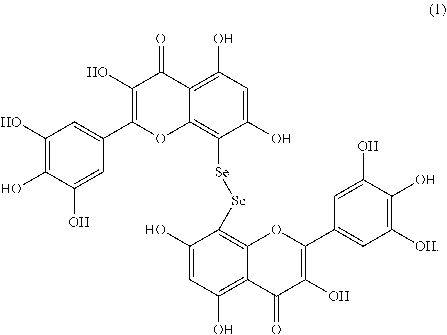 [SMILES: O=c1c(O)c(-c2cc(O)c(O)c(O)c2)oc2c([Se][Se]c3c(O)cc(O)c4c(=O)c(O)c(-c5cc(O)c(O)c(O)c5)oc34)c(O)cc(O)c12]

Furthermore, in the application provided by the present disclosure, it may also have such characteristics: the tumor includes liver cancer, lung cancer, primary colorectal cancer, cervical squamous cell carcinoma, gastric cancer, prostate cancer, and lung adenocarcinoma.

### BRIEF DESCRIPTION OF THE DRAWINGS

FIG. 1 is a calculation diagram of the IC50 value of dimyricetin-based diselenide on human gastric cancer cells in an experimental example of the present disclosure;

FIG. 2 is a calculation diagram of the IC50 value of dimyricetin-based diselenide on human liver cancer cells in an embodiment of the present disclosure;

FIG. 3 is a calculation diagram of the IC50 value of dimyricetin-based diselenide on primary colorectal cancer cells in an embodiment of the present disclosure;

FIG. 4 is a calculation diagram of the IC50 value of dimyricetin-based diselenide on human cervical squamous cell carcinoma cells in an embodiment of the present disclosure;

FIG. 5 is a calculation diagram of the IC50 value of dimyricetin-based diselenide on human lung cancer cells in an embodiment of the present disclosure;

FIG. 6 is a calculation diagram of the IC50 value of dimyricetin-based diselenide on human lung cancer cells in an embodiment of the present disclosure;

FIG. 7 is a calculation diagram of the IC50 value of dimyricetin-based diselenide on human lung adenocarcinoma cells in an embodiment of the present disclosure;

FIG. 8 is a calculation diagram of the IC50 value of cisplatin on human gastric cancer cells in an experimental example of the present disclosure;

FIG. 9 is a calculation diagram of the IC50 value of cisplatin on human liver cancer cells in an embodiment of the present disclosure;

FIG. 10 is a calculation diagram of the IC50 value of cisplatin against primary colorectal cancer cells in an embodiment of the present disclosure;

FIG. 11 is a calculation diagram of the IC50 value of cisplatin on human cervical squamous cell carcinoma cells in an embodiment of the present disclosure;

FIG. 12 is a calculation diagram of the IC50 value of cisplatin on human lung cancer cells in an embodiment of the present disclosure;

FIG. 13 is a calculation diagram of the IC50 value of cisplatin on human prostate cancer cells in an embodiment of the present disclosure;

FIG. 14 is a calculation diagram of the IC50 value of cisplatin on human lung adenocarcinoma cells in an embodiment of the present disclosure;

FIG. 15 is a calculation diagram of the IC50 value of dimyricetin-based diselenide against the new coronavirus (2019-nCoV) in the embodiment of the present disclosure.

The present disclosure provides the following advantages:

The dimyricetin-based diselenide involved in the present disclosure has the effects of treating tumors and resistance of new coronavirus. For 7 different kinds of human tumor cells, namely human liver cancer cells, human lung cancer cells (large cell lung cancer), primary colorectal cancer cells, human cervical squamous cell carcinoma cells, human gastric cancer cells, human prostate cancer cells, and human lung glands Cancer cells (pleural effusion) have a significant inhibitory effect. The IC50 value of M^{pro}, the target of the new coronavirus (2019-nCoV), is 0.807±0.0830 μM, which has a significant effect.

### DETAILED DESCRIPTIONS OF EMBODIMENTS

In order to make it easy to understand the technical means, creative features, objectives and effects achieved by the present disclosure, the pharmaceutical application of the dimyricetin-based diselenide of the present disclosure will be specifically described below in conjunction with examples.

Dimyricetin-based diselenide, molecular formula: C₃₀H₁₈O₁₆Se₂, molecular weight: 792.37, properties: yellow powder, Chinese name: dimyricetin-yl-diselenide, English name: Dimyricetin-yl-diselenide, Chinese chemical name: 8,8′-Diselenide (3,5,7-trihydroxy-2-(3,4,5-trihydroxyphenyl)-4H-1-benzo furan-4-one), English chemical name: 8,8′-diselanediylbis(3,5,7-trihydroxy-2-(3,4,5-trihydroxyphenyl)-4H-1-benzopyran-4-one), CAS registration number: N/A, the molecular structure formula is as follows:

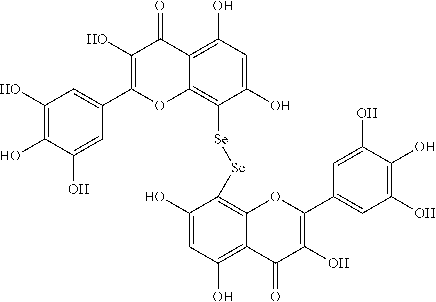 [SMILES: O=c1c(O)c(-c2cc(O)c(O)c(O)c2)oc2c([Se][Se]c3c(O)cc(O)c4c(=O)c(O)c(-c5cc(O)c(O)c(O)c5)oc34)c(O)cc(O)c12]

Dimyricetin-based diselenide has the effect of treating tumors and resistance of new coronavirus (2019-nCoV).

### Experimental Example 1

This example is a study on the efficacy of dimyricetin-based diselenide products in in vitro screening of anti-tumor drugs.

The required experimental equipment is shown in Table 1.

TABLE 1
Experimental equipment

| Name | Brand | Type |
|---|---|---|
| Petri dish | CORNING | 430599 |
| Centrifuge tube | CORNING | 430790 |
| 96-well plates | CORNING | 3599 |
| Centrifuge | DLAB | DMO412S |
| CO2 incubator | Panosonic | MCO-18AC |
| Inverted fluorescence microscope | NIKON | ECLIPSE Ti-2 |
| Clean bench | AIRTECH | SW-CJ-1FD |
| Electronic balance | METTLER | ME104E |
| | TOLEDO | |
| Inverted white light microscope | uop | DSZ2000X |
| Autoclave | Panasonic | MLS-3781L |
| Microplate reader | bio-teck | Synergy H1 |
| Thermostat Water Bath Tank | Changzhou | HH-2 |
| | Aohua | |
| refrigerator | Siemens | BCD-265 |
| | | (KG28EV2SOC) |

The required reagents are shown in Table 2.

TABLE 2
Main reagents

| Reagent | Brand | Item No. |
|---|---|---|
| MEM/EBSS | HyClone | SH30024.01 |
| RPMI-1640 | HyClone | SH30809.01 |
| DMEM/HIGH GLUCOSE | HyClone | SH30022.01 |
| F-12K Nutrient Mixture(1x) | gibco | 21127-022 |
| FBS | GIBCO | 42F3371K |
| P/S | SERVICEBIO | G4003 |
| Pancreatin | SERVICEBIO | G4001 |
| PBS | HYCLONE | SH30256.01 |

The experimental cells used are shown in Table 3.

TABLE 3
Experimental cells

| | | | STR appraisal | Tissue | |
|---|---|---|---|---|---|
| Chinese name | English name | Introduction source | report | source | Culture medium |
| Human liver cancer cells | HEP3B2.1.7 | procell | Having STR | liver | MEM-EBSS + 10% |
| | | | appraisal report | | FBS |
| Human lung cancer cells | NCI-H460 | procell | Having STR | lung | 90%RPMI-1640 + |
| (large cell lung cancer) | | | appraisal report | | 10% FBS |
| Primary colorectal cancer | SW480 | procell | Having STR | intestinal | H-DMEM + 10% |
| | | | appraisal report | | FBS |
| Human cervical squamous | Siha | ATCC | Having STR | uterus | DMEM-H + 10% |
| cell carcinoma | | | appraisal report | | FBS |
| Human gastric cancer | MGC80-3(MGC-803) | Procell's other key | Having STR | stomach | 1640 + 10% FBS |
| | | laboratories | appraisal report | | |
| Human prostate cancer cells | PC-3 | procellL | Having STR | prostate | F12 + 10% FBS |
| | | | appraisal report | | |
| Human lung adenocarcinoma | Calu-3 | procell | Having STR | lung | MEM-EBSS + 10% |
| cells (pleural effusion) | | | appraisal report | | FBS |

### Experimental Method

In vitro screening of the efficacy of dimyricetin-based diselenide on 7 different human tumor cells (HEP3B2.1.7, NCI-H460, CALU-3, SW480, SIHA, PC-3, MGC80-3) was performed. Using the CCK-8 detection method, 10 drug concentrations was designed, and each concentration was arranged in 3 re-holes. 1 detection time points, 24 h, 48 h, and 72 h, was selected according to dynamic microscopy. The positive drug was cisplatin. Cisplatin concentration gradient designed: 0 (i.e., the solvent control group), 1 μM, 5 μM, 10 μM, 20 μM, 40 μM, 60 μM, 80 μM, 100 μM, 135 μM. Dimyricetin-based diselenide drug concentration gradient designed: 0 (i.e., solvent control group), 1 μg/ml, 5 μg/ml, 25 μg/ml, 50 μg/ml, 75 μg/ml, 100 μg/ml, 125 μg/ml, 150 μg/ml, 200 μg/ml.

Specific steps are as follows:

Step 1. Cell culture. Step 1-1, the cell status under the microscope was observed, the cells were free of contamination, and the cell confluence was about 90%; Step 1-2, the supernatant was discarded, 5 mL PBS to each dish was added and washed, 3 mL trypsin was added, and digested for 3 minutes; or the liquid was changed to 5 mL. Steps 1-3, the cells were gently blew, the cells in the centrifuge tube were collected, and centrifuged at 1000 rpm for 5 min; Steps 1-4, the supernatant was discarded, part of the cells in fresh medium was resuspended and transferred to a new cell culture dish to expand the culture, the passaging ratio was 3:8. Steps 1-5, according to the experimental plan, passaging was continuing and the culture was further expanded. The confluence was about 90%.

Step 2. Drug preparation. 30.28 mg of dimyricetin-based diselenide was weighed and dissolved with DMSO. Adding of DMSO started from 100 ul. If it is not completely dissolved, 100 ul of DMSO was added again until completely dissolved to obtain the mother liquor, which is diluted to the working concentration. 50 mg of cisplatin was weighed and dissolved with DMF. Adding of DMF, started from 100 ul. If it is not completely dissolved, 100 ul DMF was added again until completely dissolved to obtain the mother liquor, which is dilute to working concentration. The working concentration is the concentration of multiple drugs designed in advance. Generally, filtration sterilization is the first choice for drug sterilization. Alternative sterilization methods include autoclaving and ultraviolet irradiation.

Step 3. Adding medicine and testing for CCK-8 plating. Step 3-1, cell seeding plate: The logarithmic growth phase cells were digested with trypsin to prepare a cell suspension, 3000-5000 cells were inoculated per well in a 96-well plate. 100 μl per well was added, and placed in CO₂ (5%) and incubated overnight at 37° C. in an incubator to adhere to the wall, and the edge holes were filled with sterile PBS. Step 3-2, the different concentrations (solvent control group, 1 ug/ml, 5 ug/ml, 25 ug/ml, 50 ug/ml, 75 ug/ml, 100 ug/ml, 125 ug/ml, 150 ug/ml, 200 ug/ml) of dimyricetin-based diselenide was added to a 96-well plate, and each sample concentration was set to 3 replicates; different concentrations (solvent control group, 1 uM, 5 uM, 10 uM, 20 uM, 40 uM, 60 uM, 80 uM, 100 uM, 135 uM) of cisplatin was added to a 96-well plate, and each sample concentration was set to 3 replicates. Step 3-3, CCK-8 reaction: 10 μl of CCK-8 solution was added to all wells, the culture plate was tapped gently to mix, and incubated in the incubator for 2 hours. Step 3-4, measuring the absorbance value: a microplate reader was used to measure the 450 nm absorbance value, and the inhibitory rate of the drug on the cells was calculated according to the formula. The formula is as follows:

Experimental group: the absorbance value of the cells and CCK-8 solution in the experimental group

Negative control: the absorbance value of control cells and CCK-8 solution

Blank control: absorbance value of medium and CCK-8 solution

Proliferation rate=(experimental group-blank control)/(negative control-blank control)×100%

Inhibition rate=1−(experimental group-blank control)/(negative control-blank control)×100%

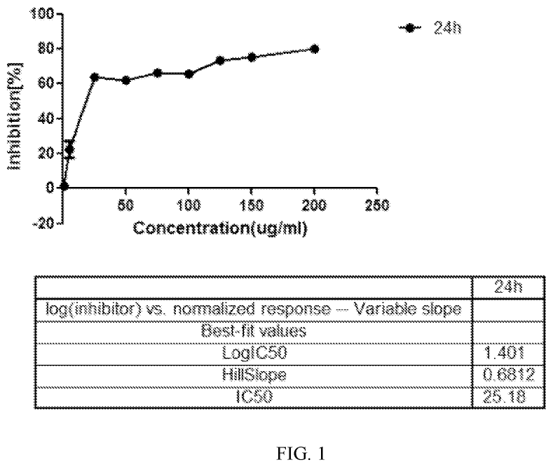

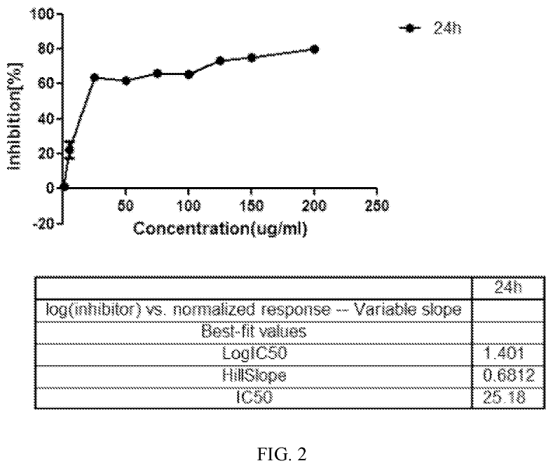

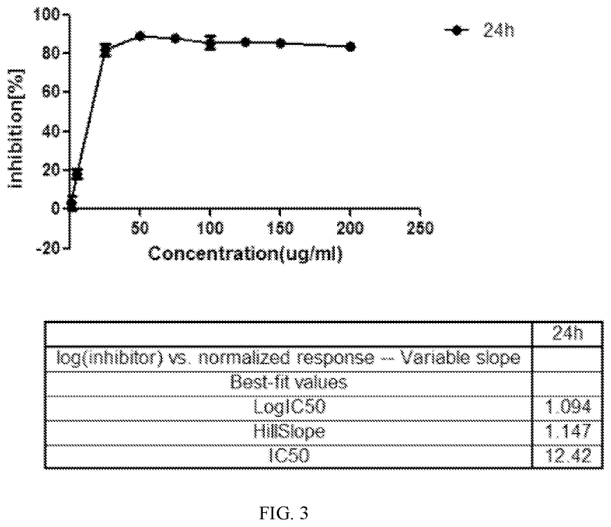

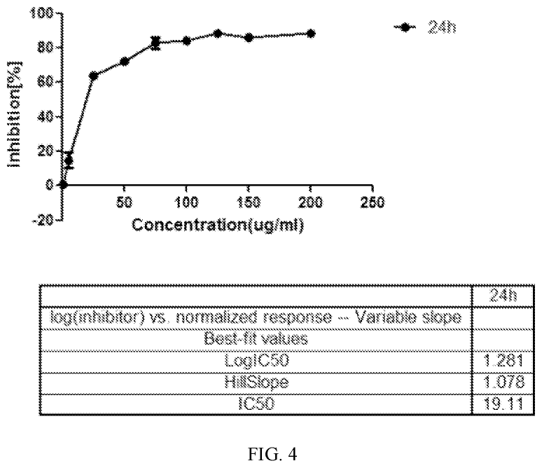

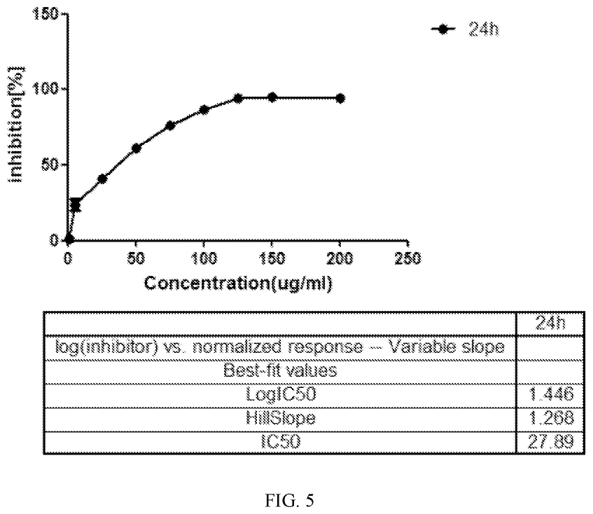

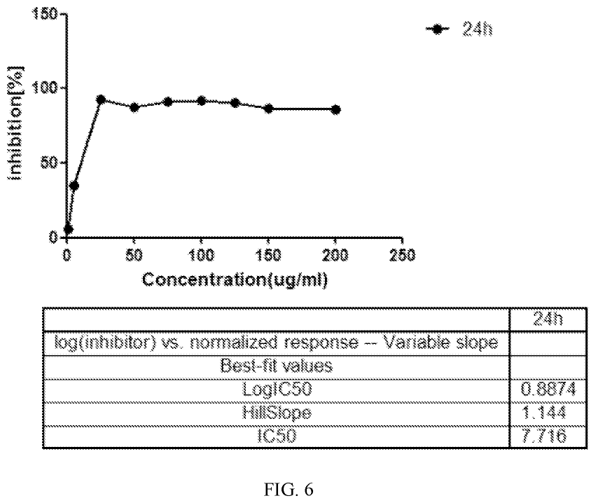

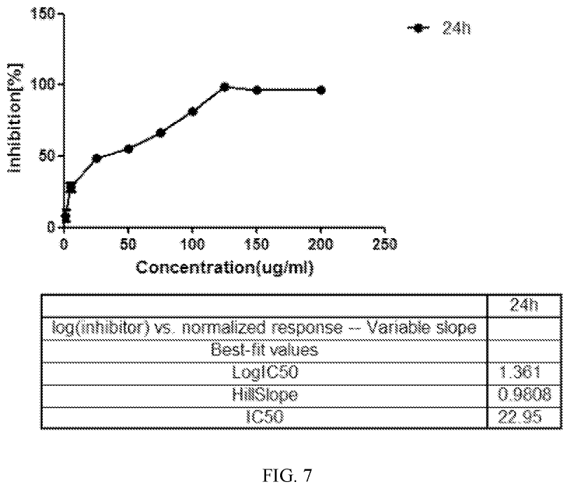

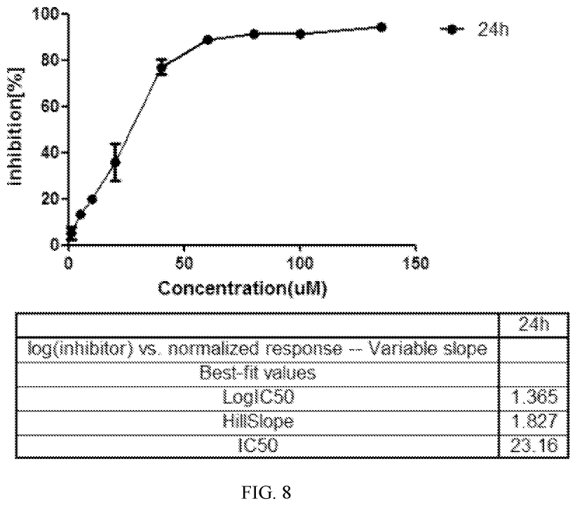

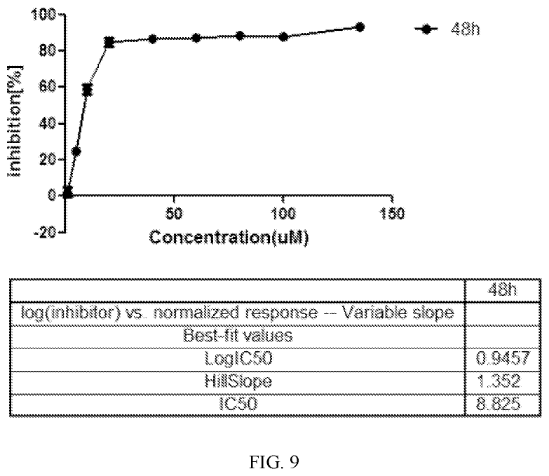

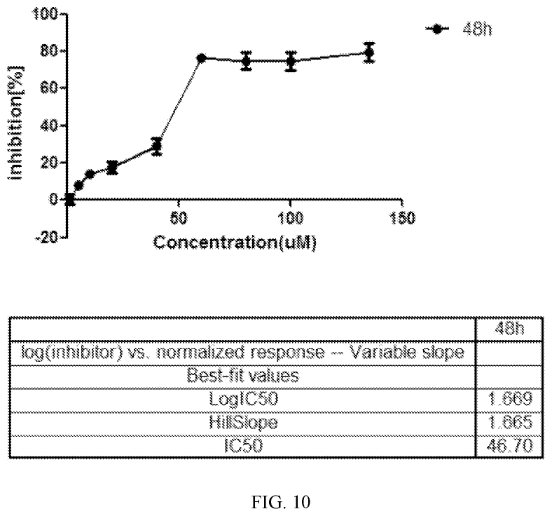

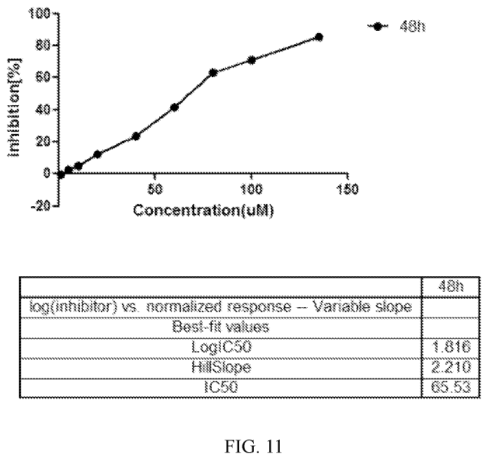

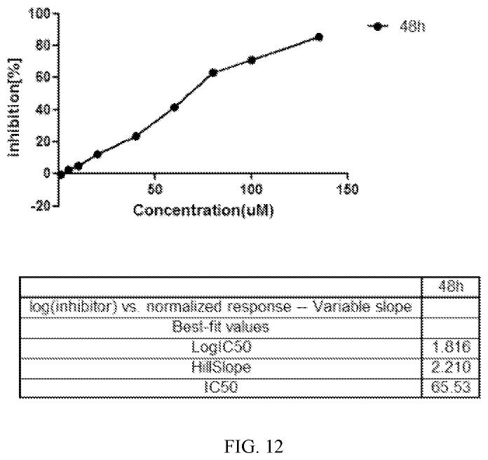

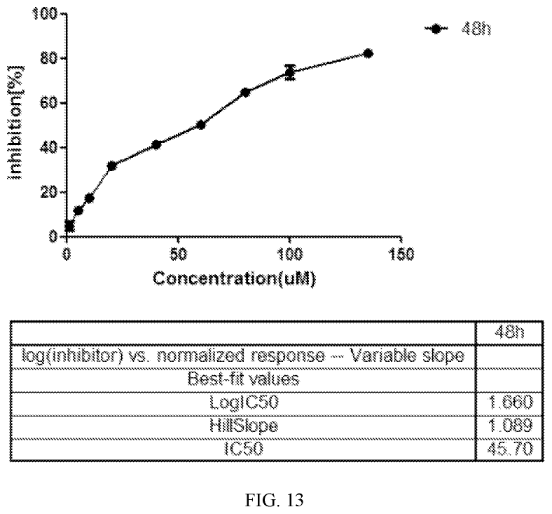

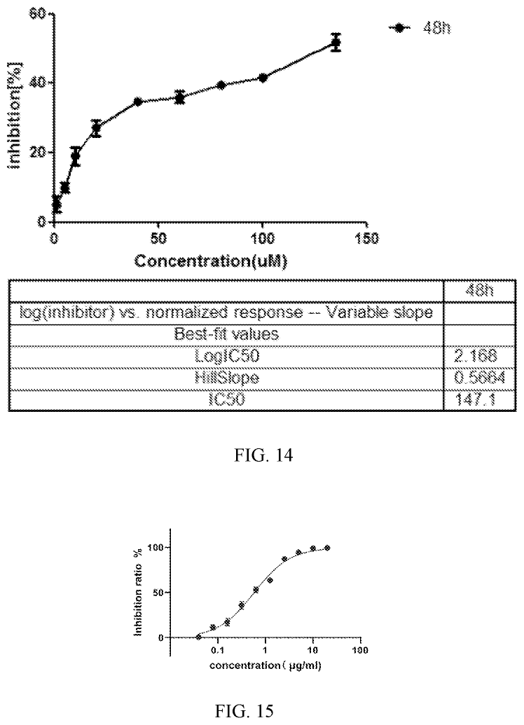

Step 4. Data analysis. Drawing with Graghpad-prism5.0, as shown in FIG. 1 to FIG. 14, the IC50 values were calculated corresponding to the time points of the 7 kinds of cells dimyricetin-based diselenide and the positive drug cisplatin.

The experimental results are shown in Table 4.

TABLE 4
The IC50 values at the time points corresponding to the 7 kinds of
cells dimyricetin-based diselenide and the positive drug cisplatin

| Cell line | MGC803 | HEP3B | sw480 | siha | H460 | pc-3 | calu-3 |
|---|---|---|---|---|---|---|---|
| Cisplatin | (24 h) | (48 h) | (48 h) | (48 h) | (48 h) | (48 h) | (48 h) |
| (μM) | 23.16 | 8.825 | 46.7 | 65.53 | 47.37 | 45.7 | 147.1 |
| Dimyricetin-based | (24 h) | (24 h) | (24 h) | (24 h) | (24 h) | (24 h) | (24 h) |
| diselenide | 9.081 | 25.18 | 12.42 | 19.11 | 27.89 | 7.716 | 22.95 |
| (μg/ml) | | | | | | | |

In this experiment: the IC50 values of all cells of dimyricetin-based diselenide are within the set drug concentration range, and the IC50 values obtained are more reliable. Except for calu-3 cells, the IC50 values of cisplatin cells are within the set drug concentration range, and the IC50 values obtained are more reliable. The IC50 value of cisplatin calu-3 cells exceeds the set drug concentration range, and the IC50 value obtained cannot be trusted.

It can be seen from Table 4 that dimyricetin-based diselenide has obvious inhibitory effects on human liver cancer cells, human lung cancer cells (large cell lung cancer), primary colorectal cancer cells, human cervical squamous cells, human gastric cancer cells, human prostate cancer cells, human lung adenocarcinoma cells (pleural effusion).

### Experimental Example 2

This example was the detection of M^{pro} protease activity inhibition of the targeted 2019-nCoV virus.

Detection principle: 3-chymotrypsin-like protease (3-chymotrypsin-like protease), the main protease (M^{pro}, also known as 3CL^{pro}), is encoded by ORF1 (located in nsp5), located in the central region of the replicase gene, and is a key protein in the replication of new coronavirus RNA. The mechanism is: after the new coronavirus invades the cell, it will use the host cell to synthesize two ultra-long replicase polypeptides (pp1a and pp1ab) necessary for self-replication. The replicase polypeptide needs to be further cut into multiple proteins (such as RdRp, helicase, etc.), and then assembled into the replication and transcription machinery required for the virus to initiate the replication of its own genetic material. M^{pro} has at least 11 cleavage sites on the replicase polypeptide. Only when these sites on the replicase polypeptide are normally cut, it is assembled into a replication transcription machine to initiate virus replication. Given that the M^{pro} protease is very important in the virus replication process, and there is no similar protein in the human body, the main protease M^{pro} has become a potential key drug target against the new coronavirus.

The fluorescence resonance energy transfer method was used to evaluate the inhibitory activity of dimyricetin-based diselenide on 2019-nCoV-M^{pro} protease.

Specific detection method: the volume of the entire enzymatic reaction system is 120 μL, the final concentration of protease is 30 nM, and the final concentration of substrate is 20 μM. The buffer of the reaction system includes 50 mM Tris pH 7.3, 1 mM EDTA. 2019-nCoV-M^{pro} protease and different concentrations of dimyricetin-based diselenide were added to a 96-well plate, incubated at 30° C. for 10 min. The substrate was added and quickly placed in the microplate reader for reading. The excitation light and emission light are 340 nm and 405 nm, respectively. The test time is 10 min, and the fluorescence value is read every 30 seconds. The final result is the first 2 minutes reading to fit the reaction rate, and compared with the control group (DMSO) to calculate the inhibition rate. Drawing with Graghpad-prism5.0, as shown in FIG. 15, the IC50 values of the 2019-nCoV virus versus dimyricetin-based diselenide corresponding to the time points.

The test results are shown in Table 5:

TABLE 5
IC50 values of 2019-nCoV virus versus
dimyricetin-based diselenide

| | IC50 (μg/ml) | IC50 (μM) |
|---|---|---|
| dimyricetin-based | 0.639 ± 0.0657 μg/ml | 0.807 ± 0.0830 μM |
| diselenide | | |

From Table 5, it can be seen that the IC50 value about dimyricetin-based diselenide against the new coronavirus (2019-nCoV) target M^{pro} is 0.807±0.0830 μM, which has a significant effect

The pharmaceutical application of the dimyricetin-based diselenide involved in the present disclosure is not limited to the scope of the specific embodiment. The above content is only a basic description of the present disclosure, and any equivalent changes made according to the technical solution of the present disclosure should belong to the protection scope of the present disclosure.

What claimed is claimed:

1. An application of dimyricetin-based diselenide represented by a molecular structure formula (1) in the treatment of tumors and resistance of new coronavirus (2019-nCoV)

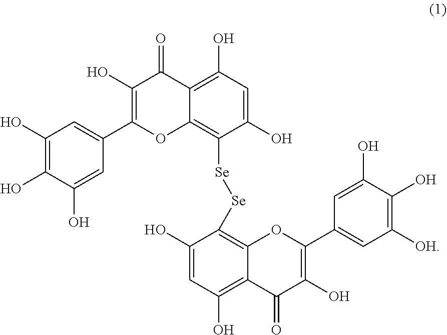 [SMILES: O=c1c(O)c(-c2cc(O)c(O)c(O)c2)oc2c([Se][Se]c3c(O)cc(O)c4c(=O)c(O)c(-c5cc(O)c(O)c(O)c5)oc34)c(O)cc(O)c12]

2. The application according to claim 1, wherein the tumor comprises liver cancer, lung cancer, primary colorectal cancer, cervical squamous cell carcinoma, gastric cancer, prostate cancer, and lung adenocarcinoma.
